(19) United States
(12) Patent Application Publication
(10) Pub. No.: US 2025/0387516 A1
(43) Pub. Date: Dec. 25, 2025

(54) MATERIALS AND METHODS FOR MODIFYING EXPRESSION OF MYOSIN HEAVY CHAIN GENES

(71) Applicant: NORTHWESTERN UNIVERSITY, Evanston, IL (US)

(72) Inventors: Anthony Martin Gacita, Chicago, IL (US); Elizabeth M. McNally, Oak Park, IL (US)

(21) Appl. No.: 18/880,416

(22) PCT Filed: Jul. 6, 2023
(86) PCT No.: PCT/US2023/069687

§ 371 Date: Dec. 31, 2024

Related U.S. Application Data
(60) Provisional application No. 63/358,994, filed on Jul. 7, 2022.

(51) Int. Cl. A61K 48/00; A61K 38/17; A61P 9/04; C12N 9/22; C12N 15/11; C12N 15/86; C12N 15/88

(52) U.S. Cl. CPC A61K 48/0058; A61K 38/1719; A61P 9/04; C12N 9/226; C12N 15/111; C12N 15/86; C12N 15/88; C07K 2319/715; C12N 2310/20; C12N 2750/14143

(57) ABSTRACT

Described herein is a method for editing the MHY7 gene in a cell by genome editing comprising introducing into the cell one or more deoxyribonucleic acid (DNA) endonucleases to effect one or more double stranded breaks (DSBs) within or near enhancer regions of the MYH7 gene or MYH6 gene that results in deletion of one or more enhancer regions of the MYH7 gene.

### FIELD OF THE INVENTION

The disclosure is directed to the use of genome editing to modify expression of myosin heavy chain (MHC) genes associated with cardiomyopathy and heart failure.

### INCORPORATION BY REFERENCE OF THE SEQUENCE LISTING

This application contains, as a separate part of disclosure, a Sequence Listing in computer-readable form (filename: 2022-044 PC_SeqListing.xml; Size: bytes: 7,227 bytes; Created: Jul. 6, 2023), which is incorporated by reference herein in its entirety.

### BACKGROUND

Cardiomyopathies are heart muscle disorders which represent a heterogeneous group of diseases that often lead to progressive heart failure with significant morbidity and mortality. Common symptoms include dyspnea, exercise and activity intolerance and peripheral oedema, and risks of having dangerous forms of irregular heart rate and sudden cardiac death are increased. The most common form of cardiomyopathy is dilated cardiomyopathy. Dilated cardiomyopathy is a heart muscle disorder characterized by dilatation and systolic dysfunction of the left or both ventricles (Elliott, P., Andersson, B., Arbustini. E., Bilinska, Z., Cecchi, F., Charron, P., Dubourg, O., Ktihl, U., Maisch, B., McKenna, W. J., et al. (2008) Classification of the cardiomyopathies: a position statement from the European society of cardiology working group on myocardial and pericardial diseases. Eur. Heart J., 29, 270-276). The ventricular walls become thin and stretched, compromising cardiac contractility and ultimately resulting in poor left ventricular function. Other forms of cardiomyopathy include hypertrophic, arrhythmogenic and restrictive. Chronic or acute coronary artery disease can also lead to ischemic cardiomyopathy and cause heart failure.

Protein coding mutations in over 100 genes have been linked to autosomal dominant cardiomyopathy, which leads to heart failure and significant burden¹⁻³. A well-recognized clinical feature of genetic cardiomyopathy is its variable phenotypic expression. Genetic cardiomyopathy demonstrates an age-dependent penetrance, variable expressivity, and variable clinical presentations, even in patients sharing identical primary mutations^{4,5}. Protein coding variants have been described as altering the phenotypic expression of primary cardiomyopathy-causing mutations⁵⁻⁷. However, the contribution of noncoding variation as modifiers of the clinical presentation of cardiomyopathy has been less well investigated.

Noncoding regions of the genome harbor important regulatory sequences that control the expression of genes through both distal enhancers and proximal gene promoters⁸. ChIP-seq, ATAC-seq, and CAGE-seq can mark genomic regions as having regulatory function, but do not provide information on their gene target. Chromatin conformation assays evaluate genomic three-dimensional organization and link enhancers to their target genes. However, as enhancer function is dependent on tissue-specific transcription factors, assays for enhancer function or targets require the context of relevant tissues/cells.

### SUMMARY

In one aspect, described herein is a method for increasing expression of the myosin heavy chain 6 (MYH6) gene in a cell comprising introducing into the cell one or more DNA endonucleases including inactivated endonucleases fused to transcriptional regulators, and one or more guide RNAs that target a region within chr14:23877406-23879693 as designated in the human genome browser, build 37 (hg37) of the MYH6 gene. In some embodiments, the method results in activation of an enhancer region of the MYH6 gene.

In another aspect, described herein is a method of improving cardiac contractility in a subject in need thereof comprising administering to the subject an agent that increases myosin heavy chain 6 (MYH6) gene expression in a cardiac cell from the subject.

### BRIEF DESCRIPTION OF THE FIGURES

FIG. 1. Genomic region directly upstream of MYH6 contains three putative enhancer regions. The H3K27Ac histone mark (“H3K27at Human LV” at the top of the Figure) indicates enhancer/promoter function. Assay for transposase-accessible chromatin (ATAC)-sequencing demonstrates (“ATAC-Seq Human LV” in the middle of the Figure) open chromatin which is a non-specific mark of regulatory function. Placental mammal sequence conservation (“Sequence Conservation”) is provided at the bottom of the Figure. Integrative analysis demonstrates three putative enhancer regions (labelled C1, C2, and C3). These regions are marked with open chromatin, sequence conservation, and are flanked by histone acetylation marks. This pattern is highly consistent with enhancer function.

FIG. 2 is a graph showing that HEK293T cells transfected with guide 1 showed the largest increase in MYH6 expression, while cells transfected with guide 2 and guide 3 showed modest increases in MYH6 expression when compared to control plasmid containing an empty sgRNA sequence as indicated by RT-qPCR.

FIG. 3 is a graph showing that human induced pluripotent stem cell cardiomyocytes (hiPSC-CMs) transfected with guide 1 showed a significant increase in MYH6 expression and hiPSC-CMs transfected with guide 2 showed a modest increase in MYH6 expression. MYH7 gene expression was not increased

### DETAILED DESCRIPTION

Inherited cardiomyopathy associates with a range of phenotypic expression. As described in the Examples, epigenomic profiling from multiple sources was superimposed, including promoter-capture chromatin conformation information, to identify candidate enhancer regions for myosin heavy chain 6 (MYH6). Enhancer function was validated in human cardiomyocytes derived from induced pluripotent stem cells and revealed enhancer regions implicated the increased expression of MYH6 and decreased expression of MYH7.

### Myosin Heavy Chain Genes, MYH6 and MYH7

In humans, both MYH7 (α-MHC) and MYH6 (β-MHC) are expressed in myocardium and cause cardiomyopathy when mutated (Carniel et al., Circulation 112:-54-59, 2005; Kamisago et al., NEJM, 343:1688-1696, 2000). These genes are in tandem on chromosome 14, with MYH6 located 5.3 kb downstream of MYH7, and their expression is developmentally regulated. MYH6 is mainly expressed in embryonic heart, whereas MYH7 becomes the predominant adult isoform (Lowes et al., J. Clin. Invest., 100:2315-2324, 1997). The adult heart retains low level expression of MYH6 (˜5%), and in the failing heart MYH6 is decreased as to become undetectable (Nakao et al., J. Clin Invest 100:2362-2370, 1997).

In one aspect, described herein is a method for increasing expression of the MHY6 gene in a cell comprising introducing into the cell one or more one or more DNA endonucleases and one or more guide RNA that target a region within or near chr14:23,877,406-23,879,693 as designated in the human genome browser, build 37 (hg37). In some embodiments, the method activates an enhancer region of the MYH6 gene. In some embodiments, the DNA endonuclease is a nuclease defective (inactive) DNA endonuclease (e.g., dCas9) as described elsewhere herein and in Gilbert et al. (Cell, 154:442-451, 2013).

In some embodiments, the enhancer region is upstream (e.g., within 500 bps) of the MYH6 gene. In some embodiments, the enhancer region is upstream within 10 bp, 20 bp, 30 bp, 40 bp, 50 bp, 60 bp, 70 bp, 80 bp, 90 bp, 100 bp, 125 bp, 150 bp, 175 bp, 200 bp, 225 bp, 250 bp, 275 bp, 300 bp, 325 bp, 350 bp, 375 bp, 400 bp, 425 bp, 450 bp, 475 bp or 500 bp of the MYH6 gene. In some embodiments, the enhancer region is MYH6-C1, MYH6-C2, or MYH6-C3. Locations of the various enhancer regions of the MYH6 gene are provided below in Table A.

TABLE A

| Region name | Genomic Location (hg19) | Size (bp) |
|---|---|---|
| MYH6 Promoter | chr14: 23,877,406-23,877,963 | 558 |
| MYH6- C1 | chr14: 23,878,449-23,878,750 | 302 |
| MYH6- C2 | chr14: 23,878,776-23,879,135 | 360 |
| MYH6- C3 | chr14: 23,879,177-23,879,693 | 517 |

In some embodiments, the method results in increased MYH6 expression in the cell, relative to a cell into which the DNA endonuclease (e.g., Cas9 or dCas9) was not introduced. In some embodiments, the method results in decreased myosin heavy chain 7 (MYH7) expression and increased MYH6 expression in the cell, relative to a cell into which the Cas9 (e.g., Cas9 or dCas9) was not introduced.

In some embodiments, the method results in at least a 10% decrease in MYH7 expression, relative to a cell into which the DNA endonuclease (e.g., Cas9 or dCas9) was not introduced. In some embodiments, the method results in at least a 15%, 20%, 25%, 30%, 35%, 40%, 50%, 55%, 60%, 65%, 70%, 75%, 80%, 85%, 90%, 95% or more decrease in MYH7 expression in a cell, relative to a cell into which the DNA endonuclease (e.g., Cas9 or dCas9) was not introduced. In some embodiments, the method results in at least a 2% increase in MYH6 expression in the cell, relative to a cell into which the DNA endonuclease (e.g., Cas9 or dCas9) was not introduced. In some embodiments, the method results in at least a 2%, 2%, 5%, 10%, 15%, 20%, 25%, 30%, 35%, 40%, 50%, 55%, 60%, 65%, 70%, 75%, 80%, 85%, 90%, 95% or more increase in MYH6 expression in a cell, relative to a cell into which the DNA endonuclease (e.g., Cas9 or dCas9) was not introduced.

### CRISPR Endonuclease System

A CRISPR (Clustered Regularly Interspaced Short Palindromic Repeats) genomic locus can be found in the genomes of many prokaryotes (e.g., bacteria and archaea). In prokaryotes, the CRISPR locus encodes products that function as a type of immune system to help defend the prokaryotes against foreign invaders, such as virus and phage. There are three stages of CRISPR locus function: integration of new sequences into the locus, biogenesis of CRISPR RNA (crRNA), and silencing of foreign invader nucleic acid. Five types of CRISPR systems (e.g., Type I, Type II, Type III, Type U, and Type V) have been identified.

A CRISPR locus includes a number of short repeating sequences referred to as “repeats.” The repeats can form hairpin structures and/or comprise unstructured single-stranded sequences. The repeats usually occur in clusters and frequently diverge between species. The repeats are regularly interspaced with unique intervening sequences referred to as “spacers,” resulting in a repeat-spacer-repeat locus architecture. The spacers are identical to or have high homology with known foreign invader sequences. A spacer-repeat unit encodes a crisprRNA (crRNA), which is processed into a mature form of the spacer-repeat unit. A crRNA comprises a “seed” or spacer sequence that is involved in targeting a target nucleic acid (in the naturally occurring form in prokaryotes, the spacer sequence targets the foreign invader nucleic acid). A spacer sequence is located at the 5′ or 3′ end of the crRNA.

A CRISPR locus also comprises polynucleotide sequences encoding CRISPR Associated (Cas) genes. Cas genes encode endonucleases involved in the biogenesis and the interference stages of crRNA function in prokaryotes. In some embodiments, the DNA endonuclease is Cas9. Examples of various Cas9 proteins and Cas9 guide RNAs (as well as information regarding requirements related to protospacer adjacent motif (PAM) sequences present in targeted nucleic acids) can be found in the art, for example, see Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al., Cell. 2013 Feb. 28; 152(5):1173-83; Wang et al., Cell. 2013 May 9; 153(4):910-8; Auer et al., Genome Res. 2013 Oct. 31; Chen et al., Nucleic Acids Res. 2013 Nov. 1; 41(20):e19; Cheng et al., Cell Res. 2013 October; 23(10):1163-71; Cho et al., Genetics. 2013 November; 195(3):1177-80; DiCarlo et al., Nucleic Acids Res. 2013 April; 41(7):4336-43; Dickinson et al., Nat Methods. 2013 October; 10(10):1028-34; Ebina et al., Sci Rep. 2013; 3:2510; Fujii et al., Nucleic Acids Res. 2013 Nov. 1; 41(20):e187; Hu et al., Cell Res. 2013 November; 23(11):1322-5; Jiang et al., Nucleic Acids Res. 2013 Nov. 1; 41(20):e188; Larson et al., Nat Protoc. 2013 November; 8(11):2180-96; Mali et al., Nat Methods. 2013 October; 10(10):957-63; Nakayama et al., Genesis. 2013 December; 51(12):835-43; Ran et al., Nat Protoc. 2013 November; 8(11):2281-308; Ran et al., Cell. 2013 Sep. 12; 154(6):1380-9; Upadhyay et al., G3 (Bethesda). 2013 Dec. 9; 3(12):2233-8; Walsh et al., Proc Natl Acad Sci USA. 2013 Sep. 24; 110(39):15514-5; Xie et al., Mol Plant. 2013 Oct. 9; Yang et al., Cell. 2013 Sep. 12; 154(6):1370-9; Briner et al., Mol Cell. 2014 Oct. 23; 56(2):333-9; and U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; all of which are hereby incorporated by reference in their entirety.

Cas genes encode endonucleases involved in the biogenesis and the interference stages of crRNA function in prokaryotes. Some Cas genes comprise homologous secondary and/or tertiary structures. In some embodiments, the DNA endonuclease is a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease; or a homolog thereof. In some embodiments, the DNA endonuclease is Cas9.

In some embodiments, the DNA endonuclease is a nuclease defective DNA endonuclease. For example, certain DNA endonuclease (e.g., Cas9) modifications can provide a nuclease that does not cleave or nick, or does not substantially cleave or nick the target sequence. Thus, a nuclease defective DNA endonuclease has been rendered inactive, and can be used in combination with one or more guide RNAs to bind to a target genomic location comprising a target gene, wherein binding to the target genomic location is dictated by the one or more guide RNAs. The binding of the DNA endonuclease and the one or more guide RNAs, optionally in combination with a transcriptional modulator (e.g., a transcriptional activator), thereby regulates transcription of the target gene. In some embodiments, the nuclease defective DNA endonuclease is a nuclease defective Cas9 (i.e., dCas9). Exemplary mutations that reduce or eliminate nuclease activity include one or more mutations in the following locations: D10, G12, G17, E762, H840, N854, N863, H982, H983, A984, D986, or A987, or a mutation in a corresponding location in a Cas9 homologue or ortholog. The mutation(s) can include substitution with any natural (e.g., alanine) or non-natural amino acid, or deletion. An exemplary nuclease defective dCas9 protein is Cas9D10A&H840A (Jinek, et al., Science. 2012 Aug. 17; 337(6096):816-21; Qi, et al., Cell. 2013 Feb. 28; 152(5):1173-83).

dCas9 proteins that do not cleave or nick the target sequence can be utilized in combination with an gRNA to form a complex that is useful for transcriptional modulation of target nucleic acids. The dCas9 can be targeted to one or more genetic elements by virtue of the binding regions encoded on one or more sgRNAs. Recruitment of dCas9 can therefore provide recruitment of additional effector functions as provided by polypeptides fused to the dCas9 domain. For example, a polypeptide comprising an effector function can be fused to the N and/or C-terminus of a dCas9 domain. In some embodiments, the polypeptide encodes a transcriptional activator or transcriptional repressor. In some embodiments, the transcriptional activator is such as VP16, VP64, synergistic activation mediator, or SunTag.

In some embodiments, the DNA endonuclease (e.g., Cas9 or dCas9) is introduced to the cell as a protein (i.e., a protein-based system). Typically, the cell is treated chemically, electrically, or mechanically to allow DNA endonuclease (e.g., Cas9 or dCas9) entry into the cell. Alternatively, in some embodiments, the DNA endonuclease (e.g., Cas9 or dCas9) is introduced to the cell as a nucleic acid (e.g., DNA or mRNA) under conditions which allow production of the nuclease. Guide RNA also is introduced into the cell.

In some embodiments, the methods described herein comprise introducing one or more guide RNAs into the cell. A genome-targeting RNA is referred to as a “guide RNA” or “gRNA” herein. A guide RNA comprises at least a spacer sequence that hybridizes to a target nucleic acid sequence of interest, and a CRISPR repeat sequence. In Type II systems, the gRNA also comprises a tracrRNA sequence. In the Type II guide RNA, the CRISPR repeat sequence and tracrRNA sequence hybridize to each other to form a duplex. The duplex binds a site-directed polypeptide, such that the guide RNA and site-direct polypeptide form a complex. The guide RNA provides target specificity to the complex by virtue of its association with the Cas9 (e.g., Cas9 or dCas9) nuclease. The guide RNA thus directs the activity of the DNA endonuclease. In some embodiments, the one or more gRNA comprises a nucleotide sequence set forth in SEQ ID NO: 1, SEQ ID NO: 2, or SEQ ID NO: 3, or combinations thereof.

In some embodiments, the methods described herein comprise delivering the DNA endonuclease (e.g., Cas9 or dCas9) and one or more gRNAs to the cell by a viral vector, lipid nanoparticles (LNPs), or a combination thereof.

In some embodiments, the methods described herein comprise delivering the DNA endonuclease (e.g., Cas9 or dCas9) and one or more gRNAs to the cell by a viral vector. Non-limiting exemplary viral vectors include adeno-associated virus (AAV) vector, lentivirus vectors, adenovirus vectors, helper dependent adenoviral vectors (HDAd), herpes simplex virus (HSV-1) vectors, bacteriophage T4, baculovirus vectors, and retrovirus vectors. In some embodiments, the viral vector may be an AAV vector. In some embodiments, the viral vector is AAV2, AAV3, AAV3B, AAV5, AAV6, AAV6.2, AAV7, AAVrh.64R1, AAVhu.37, AAVrh.8, AAVrh.32.33, AAV8, AAV9, AAVrh10, or AAVLK03. In other embodiments, the viral vector may a lentivirus vector.

In some embodiments, a viral vector comprises one or more transcription and/or translation control elements. Depending on the host/vector system utilized, any of a number of suitable transcription and translation control elements, including constitutive and inducible promoters, transcription enhancer elements, transcription terminators, etc., may be used. In some embodiments, the promoter may be constitutive, inducible, or tissue-specific. In some embodiments, the promoter may be a constitutive promoter. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EF1a promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech). In some embodiments, when a TetOn promoter is utilized, the cell is also contacted with a tetracycline transactivator. In some embodiments, the promoter is a tissue-specific promoter. In some embodiments, the tissue-specific promoter is TNNT2, MLC2v, creatine kinase (CK) and derivatives thereof.

The DNA endonuclease (e.g., Cas9 or dCas9)-encoding nucleic acid is operably linked to a promoter that drives protein expression. For expressing small RNAs, including guide RNAs used in connection with Cas or Cpf1 endonuclease, promoters such as RNA polymerase III promoters, including for example U6 and H1, can be advantageous. Suitable promoters, as well as parameters for enhancing the use of such promoters, are known in art, and additional information and approaches are regularly being described; see, e.g., Ma, H. et al., Molecular Therapy—Nucleic Acids 3, e161 (2014) doi:10.1038/mtna.2014.12.

In some embodiments, the nucleotide sequence encoding the guide RNA may be located on the same vector comprising the nucleotide sequence encoding the Cas9 (e.g., Cas9 or dCas9). In some embodiments, expression of the guide RNA and of the Cas9 (e.g., Cas9 or dCas9) may be driven by their own corresponding promoters. In some embodiments, expression of the guide RNA may be driven by the same promoter that drives expression of the Cas9 (e.g., Cas9 or dCas9). In some embodiments, the guide RNA and the Cas9 (e.g., Cas9 or dCas9) transcript may be contained within a single transcript. For example, the guide RNA may be within an untranslated region (UTR) of the endonuclease transcript. In some embodiments, the guide RNA may be within the 5′ UTR of the transcript. In other embodiments, the guide RNA may be within the 3′ UTR of the transcript.

In some embodiments, the methods described herein comprise delivering the DNA endonuclease (e.g., Cas9 or dCas9) and one or more gRNAs to the cell by a lipid nanoparticle. Lipid nanoparticles include, but are not limited to, liposomes and micelles. Any number of lipids may be present, including cationic and/or ionizable lipids, anionic lipids, neutral lipids, amphipathic lipids, conjugated lipids (e.g., PEGylated lipids), and/or structural lipids. Such lipids can be used alone or in combination.

Nanoparticles are ultrafine particles typically ranging between 1 and 100 to 500 nanometers (nm) in size with a surrounding interfacial layer and often exhibiting a size-related or size-dependent property. Nanoparticle compositions are myriad and encompass lipid nanoparticles (LNPs), liposomes (e.g., lipid vesicles), and lipoplexes. For example, a nanoparticle composition can be a liposome having a lipid bilayer with a diameter of 500 nm or less. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In some embodiments, the DNA endonuclease (e.g., Cas9 or dCas9) and gRNA(s) are each separately formulated for delivery, e.g., in lipid nanoparticles. In some embodiments, the DNA endonuclease (e.g., Cas9 or dCas9) and gRNA(s) are co-formulated for delivery, e.g., in a single lipid nanoparticle. In some embodiments, an expression vector encoding the DNA endonuclease (e.g., Cas9 or dCas9) and an expression vector encoding the gRNA(s) are separately formulated for delivery, e.g., in lipid nanoparticles. In some embodiments, an expression vector encoding the DNA endonuclease (e.g., Cas9 or dCas9) and an expression vector encoding the gRNA(s) are co-formulated for delivery, e.g., in a single lipid nanoparticle.

### Treatment Methods

Thus, in another aspect, the disclosure provides a method for improving heart function in a subject in need thereof comprising administering to the subject an agent that increases MYH6 or to increase MYH6 gene expression relative to MYH7 gene expression in a cardiac cell of the subject. In some embodiments, the agent increases MYH6 gene expression by at least 3% (e.g., 3%, 4%, 5%, 6%, 7%, 8%, 9%, 10% or more) with no change in the amount of MYH7 gene expression in a cardiac cell of the subject. In some embodiments, the agent increases MYH6 gene expression by at least 3% (e.g., 3%, 4%, 5%, 6%, 7%, 8%, 9%, 10% or more) and decreases MYH7 gene expression by at least 5% (e.g., 5%, 6%, 7%, 8%, 9%, 10% or more) in a cardiac cell of the subject.

In some embodiments, the subject is suffering from cardiomyopathy, heart failure, arrhythmia, ischemic heart disease, non-ischemic heart disease and exercise or activity intolerance, and where alternatives may require heart transplantation or ventricular assist device.

As used herein, “cardiomyopathy” refers to any disease or dysfunction of the myocardium (heart muscle) in which the heart is abnormally enlarged, thickened and/or stiffened. As a result, the heart muscle's ability to pump blood is usually weakened and unable to meet the demands of the body, often leading to congestive heart failure. The disease or disorder can be, for example, inflammatory, metabolic, toxic, infiltrative, fibrotic, hematological, genetic, or unknown in origin. Such cardiomyopathies may result from a lack of oxygen. Other diseases include those that result from myocardial injury which involves damage to the muscle or the myocardium in the wall of the heart as a result of disease or trauma. Myocardial injury can be attributed to many things such as, but not limited to, cardiomyopathy, myocardial infarction, or congenital heart disease. The cardiac disorder may be pediatric in origin. Cardiomyopathy includes, but is not limited to, cardiomyopathy (dilated, hypertrophic, restrictive, arrhythmogenic, ischemic, genetic, idiopathic and unclassified cardiomyopathy), sporadic dilated cardiomyopathy, X-linked Dilated Cardiomyopathy (XLDC), acute and chronic heart failure, right heart failure, left heart failure, biventricular heart failure, congenital heart defects, myocardial fibrosis, mitral valve stenosis, mitral valve insufficiency, aortic valve stenosis, aortic valve insufficiency, tricuspidal valve stenosis, tricuspidal valve insufficiency, pulmonal valve stenosis, pulmonal valve insufficiency, combined valve defects, myocarditis, acute myocarditis, chronic myocarditis, viral myocarditis, diastolic heart failure, heart failure with reduced ejection fraction, heart failure with moderately reduced ejection fraction, heart failure with preserved ejection fraction, systolic heart failure, diabetic heart failure and accumulation diseases. In some embodiments, the heart failure includes reduced ejection fraction. In further embodiments, the heart failure includes preserved ejection fraction.

In some embodiments, the subject is suffering from heart failure. The term “heart failure” as used herein refers to a condition that develops when the heart, via an abnormality of cardiac function (detectable or not), fails to pump blood at a rate commensurate with the requirements of the metabolizing tissues or is able to do so only with an elevated diastolic filling pressure.

In some embodiments, the methods comprise treating heart failure in the subject. It will be appreciated that “treating heart failure” does not require complete amelioration of heart failure; “treating” includes any improvement in a symptom or manifestation of heart failure that confers a beneficial effect on the subject.

In some embodiments, the methods result in an improvement in cardiac function. Methods for measuring cardiac function (e.g., contractile function) are known in the art and are described, for example, in the Textbook of Medical Physiology, Tenth edition, (Guyton et al., W.B. Saunders Co., 2000). For example, cardiac ejection can be monitored using, e.g., echocardiography, nuclear or radiocontrast ventriculography, or magnetic resonance imaging. Other measures of cardiac function include, but are not limited to, myocardial contractility, resting stroke volume, resting heart rate, resting cardiac index, Doppler imaging, cardiovascular performance during stress/exercise. Optionally, cardiac function is increased by at least 2%, 5%, 10%, 15%, 20%, 25%, 30%, 40%, 50%, 60%, 70%, 80%, 90%, or even 100% relative to the cardiac function prior to treatment.

In some embodiments, the method results in an improvement in contractility of a cardiac cell, relative to a cell into which the DNA endonuclease (e.g., Cas9 or dCas9) was not introduced. In some embodiments, the method results in at least a 2% improvement in cell contractility, relative to a cell into which the DNA endonuclease (e.g., Cas9 or dCas9) was not introduced. In some embodiments, the method results in at least a 2%, 3%, 5%, 10%, 15%, 20%, 25%, 30%, 35%, 40%, 50%, 55%, 60%, 65%, 70%, 75%, 80%, 85%, 90%, 95% improvement in cell contractility, relative to a cell into which the DNA endonuclease (e.g., Cas9 or dCas9) was not introduced. Methods of measuring cell contraction include, but are not limited to, echocardiography, IPSC-derived cardiomyocyte engineered heart tissue imaging, cardiac magnetic resonance imaging (MRI), computed tomography (CT), nuclear magnetic resonance (NMR), and positron emission tomography (PET) or increased function in engineered heart tissues derived from induced pluripotent stem cells.

In some embodiments, the method partially rescues or improves one or more of the following: ejection fraction; left ventricle wall thickness; right ventricle wall thickness; left ventricular wall stress; right ventricular wall stress; ventricular mass; contractile function; cardiac hypertrophy; end diastolic volume; end systolic volume; cardiac output; cardiac index; pulmonary capillary wedge pressure; pulmonary artery pressure; 6 minute walk distance or time, performance on exercise testing, increase in ambulatory activity as monitored remotely by an activity monitor; reduction in serum biomarkers such as N-terminal pro BNP or troponin; and improvement in kidney function as it related to improve blood flow to the kidney.

Treating cardiomyopathy or heart failure in this embodiment would be undertaken to eliminate or postpone need for mechanical support of heart function such as use of a ventricular assist device and/or cardiac transplantation.

All of the U.S. patents, U.S. patent application publications, U.S. patent applications, foreign patents, foreign patent applications and non-patent publications referred to in this specification, are incorporated herein by reference, in their entireties.

From the foregoing it will be appreciated that, although specific embodiments of the invention have been described herein for purposes of illustration, various modifications may be made without deviating from the spirit and scope of the invention.

### EXAMPLES

### Example 1—Materials and Methods

Epigenetic Datasets: For histone ChIP-Seq datasets and ATAC-seq datasets, the “fold change over negative control” bigwig file was used. For transcription factor Chip-seq datasets, peak bed files were used. For Homer computational predictions, a bed file representing the location of the transcription factor motif genome-wide was used. Files were imported into the UCSC genome browser for visualization. When necessary, datasets from mouse cells/tissues or hg38 were overlaid to hg19 using the UCSC liftover tool. For pcHiC data, the CHiCAGO pipeline raw output of three replicates of IPSC-CM promoter capture Hi-C data were downloaded.¹ Probe-probe interactions were filtered. 1 kb was added to both ends of regions interacting with gene promoters. Data from each replicate was intersected using bedtools and retained only genomic interactions that were present in at least two replicates.² Bed files representing pcHi-C interactions were visualized in the UCSC genome browser.

Epigenetic Dataset Downloads and Visualization. Epigenetic datasets were identified from the Encode data repository or GEO. For histone ChIP-Seq datasets and ATAC-seq datasets, the “fold change over negative control” bigwig file was downloaded. For transcription factor Chip-seq datasets, peak bed files were downloaded. For Homer computational predictions, a bed file representing the location of the transcription factor motif genome-wide was downloaded. Files were imported into the UCSC genome browser for visualization. When necessary, datasets from mouse cells/tissues or hg38 were overlaid to hg19 using the UCSC liftover tool.

For pcHiC data, the CHiCAGO pipeline raw output of three replicates of IPSC-CM promoter capture Hi-C data were downloaded¹⁰. Probe-probe interactions were filtered. 1 kb was added to both ends of regions interacting with gene promoters. Data was intersected from each replicate using bedtools and retained only genomic interactions that were present in at least two replicates³⁰. Bed files representing pcHi-C interactions were visualized in the UCSC genome browser.

Enhancer Region Cloning. Candidate enhancer regions were ligated into luciferase plasmids using a Gateway cloning strategy. Candidate enhancer regions were amplified from human genomic DNA using primers with a 5′-CACC overhang using Phusion High-Fidelity DNA polymerase (NEB). An aliquot of the PCR reaction was separated on a 1% agarose-TBE gel to confirm amplification, and the remaining reaction was purified using a PCR Purification Kit (Qiagen). In cases where PCR failed to generate an adequate product, the enhancer region sequence (matching hg19) was synthesized as a dsDNA gGlock gene fragment (IDT). Approximately 5 ng of PCR product or gBlock was ligated into the pENTR/D-TOPO vector following manufacturer's instructions (ThermoFisher). The enhancer region was recombined into pGL4.23-GW (Addgene #60323) using LR Clonase II Enzyme mix (Thermo) with 150 ng of each plasmid. EndoFree Maxipreps (Qiagen) were used to prepare DNA. Plasmids were confirmed using Sanger Sequencing.

Enhancer constructs: Candidate enhancer regions were amplified from human genomic DNA using primers with a 5′-CACC overhang using Phusion High-Fidelity DNA polymerase (NEB). An aliquot of the PCR reaction was separated on a 1% agarose-TBE gel to confirm amplification, and the remaining reaction was purified using a PCR Purification Kit (Qiagen). In cases where PCR failed to generate an adequate product, the enhancer region sequence (matching hg19) was synthesized as a dsDNA gGlock gene fragment (IDT). Approximately 5 ng of PCR product or gBlock was ligated into the pENTR/D-TOPO vector following manufacturer's instructions (ThermoFisher). The enhancer region was recombined into pGL4.23-GW (Addgene #60323) using LR Clonase II Enzyme mix (Thermo) with 150 ng of each plasmid. EndoFree Maxipreps (Qiagen) were used to prepare DNA. Plasmids were confirmed using Sanger Sequencing. In all candidate enhancer plasmids, the enhancer sequence was located 125 bp upstream of the minimal promoter sequence. Candidate enhancer regions are shown below in Table 1.

| Region Name | Size (bp) | Coordinates (hg19) |
|---|---|---|
| MYH6- C1 | 302 | chr14: 23,878,449-23,878,750 |
| MYH6- C2 | 360 | chr14: 23,878,776-23,879,135 |
| MYH6- C3 | 517 | chr14: 23,879,177-23,879,693 |

Negative distances refer to upstream the transcriptional start site (TSS) and positive distances are downstream.

Luciferase Reporter Assay. HL-1 cardiomyocytes (Millipore Sigma Cat #SCC065) are cultured on fibronectin coated flasks in Claycomb media with 10% HL-1 qualified FBS as previously described.³¹ Twenty-four hours before transfection, 140,000 HL-1 cells per well are plated on to a 12-well plate. On the day of transfection, HL-1 cells are transfected using Lipofecamine 3000 (Thermo Fisher) following manufacturer's instructions. Each well is transfected with 6 μl of 0.15 μM enhancer firefly luciferase plasmid, 50 ng of pRL-SV40 (Promega), 2.5 μl of Lipofecamine3000, and 6 μl of P3000 in 100 μl of Opti-MEM. Cells are allowed to incubate for 6-8 hours, following which half the media was replaced with Claycomb media. Forty-eight hours after transfection, the luciferase assay is performed with the Dual-Glo luciferase assay kit (Promega) according to manufacturer's instructions. The firefly luciferase signal from each well is recorded from three separate replicates and internally normalized to Renilla luciferase signal. Each enhancer construct is tested in a minimum of two separate wells on three separate days.

Induced pluripotent stem cell (iPSC)-derived cardiomyocytes (iPSC-CMs) are generated according to standard protocols³². At approximately day 10 of differentiation, cardiomyocytes are re-plated on to white clear-bottom 96-well plates at 40,000 cells per well. The media is changed every two days and cells began to beat as a syncytium day 14-16. On day 18, cardiomyocytes are transfected with Lipofecamine3000 (Thermo Fisher) according to manufacturer's instructions. Each well is transfected with 0.2 μl of 0.1 μM enhancer firefly luciferase plasmid, 5 ng of pRL-SV40 (Promega), 0.15 μl Lipofecamine3000, and 0.2 μl of P3000 in 10 μl of Opti-MEM. Forty-eight hours after transfection, the luciferase assay is performed with the Dual-Glo luciferase assay kit (Promega) according to manufacturer's instructions. Firefly luciferase signal was read using 96-well plate reader and signals were internally normalized to the same well's Renilla luciferase signal. Each enhancer construct is tested in 8 separate wells on at least three separate cardiomyocyte differentiations.

IPSC Reprogramming, Culturing, and IPSC-CM Differentiation. Human skin fibroblasts are obtained from Coriell (sample name GM03348, 10 year old male) and cultured in DMEM containing 10% FBS. Fibroblasts are re-programmed into induced pluripotent stem cells (IPSCs) via electroporation with pCXLE-hOCT3/4-shp53-F (Addgene plasmid 27077), pCXLE-hSK (Addgene plasmid 27078), and pCXLE-hUL (Addgene plasmid 27080) as described previously³³. IPSCs are maintained on Matrigel-coated 6-well plates with mTeSR-1 (Stem Cell technologies, Cat #85850) and passaged as colonies every 5-7 days using ReLeSR (Stem Cell technologies, Cat #05872).

IPSCs are differentiated into cardiomyocytes (iPSC-CMs) using Wnt modulation as previously described³². Differentiation is conducted in CDM3 (RPMI 1640 with L-glutamine, 213 μg/mL L-asorbic acid 2-phosphate, 500 μg/mL recombinant human albumin)³². Cells are grown to approximately 95% confluency and treated with 6 μM-10 μM CHIR99021 for 24 hours and allowed to recover for 24 hours. Cells are treated with 2 μM Wnt-C59 for 48 hours and then media is changed with CDM3 every two days until beating cardiomyocytes were obtained (approximately day 6-10). In order to prevent cell detachment, beating cardiomyocytes are re-plated on to new plates using TrypLE (Thermo Fisher). Media is changed every two days until downstream assays were performed (˜day 20).

CRISPr Enhancer Deletion in IPSCs. To delete enhancer regions, guides targeting the 5′ and 3′ end of MYH6C1-C3 enhancer regions are designed using CRISPR³⁴.

Guides are ligated into pSpCas9(BB)-2A-Puro (Addgene plasmid #62988) after the U6 promoter using either Bbs1 digestion and ligation or Gibson assembly. DNA preparations of plasmid ae prepared using an EndoFree plasmid kit (Qiagen), and plasmid sequences are confirmed with Sanger sequencing. IPSCs are nucleofected using the Neon transfection system (Thermo Fisher). Briefly, GM03348 IPSCs are grown to approximately 70% confluency and treated with mTeSR-1 containing 2 μM thiazovivin (TZV) for one hour. Cells are digested with TrypLE, collected and counted. 3.75 million IPSCs per nucleofection are pelleted at 300 g for 3 min. Cell pellets are resuspended in 125 μl of buffer R and added to an Eppendorf tube containing 1.5 μg or 2.5 μg of each plasmid. Cells are nucleofected in the Neon system in a 100 μl tip with the following settings: 1400 V, 20 ms, 2 pulses. Nucleofected cells are expelled into a single well of Matrigel-coated 6-well plate containing mTeSR-1 supplemented with ClonR (Stem Cell Technologies, Cat #05888) and 2 μM TZV. For each round, a pSpCas9(BB)-2A-GFP (Addgene plasmid #48138) control is included. Twenty-four hours later, cells are treated with mTeSR-1 containing 0.15 μg/mL puromycin. The next day, selection is continued with 0.2 μg/mL puromycin until no viable cells are seen in the GFP control (approximately 2-3 days). Cells are switched to mTeSR-1 supplemented with ClonR and 2 μM TZV and media is changed daily until colonies appeared (5-7 days). Colonies are picked on to 96-well plates, expanded, and split on to two duplicate plates. The first plate is used for cryopreservation in 50% mTeSR-1/ClonR/2 μM TZV and 50% KnockOut Serum replacement/25% DMSO. The second plate is processed for gDNA isolation using the DirectPCR lysis reagent (Viagen, Cat #301-C) following manufacturer's instructions. Colonies are screened for successful enhancer deletion using a 3-primer PCR approach. PCR products are cloned using the TOPO TA cloning kit (Thermo Fisher) and sequenced to determine alleles present. Positive colonies are thawed from the frozen plate, expanded, re-genotyped, and used for differentiation. In cases where no homozygous deletions are obtained, a heterozygous colony is treated with a second round of CRISPR editing.

IPSC Chromosome Analysis and CRIPSr-Off Target Analysis. IPSC Chromosome analysis is conducted using the hPSC genetic analysis kit (Stem Cell Technologies, Cat #07550) following manufacturer's instructions. IPSC lines must show no amplification or deletion in at least 8 of the 9 tested sites to pass the karyotypic quality control standards. The output from the CRISPOR³⁴ guide design tool is used to identify the most likely off target cut sites. Any regions with <3 mismatches and additional off targets that are within or near genes important for cardiac function are selected. Primers are designed to amplify putative off target sites and regions are amplified from gene edited cell gDNA. PCR products are purified using ExoSAp-IT (Thermo) or Ampure XP beads (Beckman Coulter) and sequenced with sanger sequencing. Sanger traces from unedited IPSCs are compared to gene edited lines to identify any off-target changes.

IPSC-CM RNA Extraction and qPCR. At ˜day 10 of differentiation, 1 million IPSC-derived cardiomyocytes are plated on a well of 12-well plate. At approximately day 20, cells were washed with PBS and 400 μl of TRIzol (Thermo Fisher) is added directly to the well. Cells are collected into an Eppendorf tube using a cell scraper. Trizol is kept at −80° C. until further processing. Six hundred μ1 of additional TRIzol is added to the cells and the entire sample is added to a tube containing 250 μl of silica-zirconium beads. Tubes are placed in a bead beater homogenizer (BioSpec) for 1 minute and immediately cooled on ice. Samples are incubated at room temperature for 5 min and then centrifuged at 12,000 g for 5 min to remove unhomogenized cell aggregates. Supernatant is transferred to a new tube and 200 μl of chloroform is added. After vigorous shaking for 30 seconds followed by 10 min incubation with periodic shaking, samples are centrifuged at 12,000 g for 15 min. The upper aqueous layer is added to an equal volume of fresh 70% ethanol and used an input to the Aurum Total RNA Mini Kit (Biorad). RNA is processed according to manufacturer's instructions including on-column DNase digestion. RNA is eluted twice with 30 μl of warmed water and the concentration is measured using a nanodrop spectrophotometer.

The qScript cDNA SuperMix (Quantabio) is used to generate a 100 ng cDNA library. A 1:10 dilution is used as a template in a 3-step SYBR-green qPCR region with a 57° C. annealing temperature. A panel of primers targeting cardiomyocyte references genes (TNNT2, MYBPC3, TNNI3, SLC8A1, MYOZ2 and GAPDH) that passed optimization studies confirming primer specificity and efficiency is used. For enhancer deletion measurements, changes in MYH6 and MYH7 expression are calculated using the delta-delta Cq method using the geometric mean expression of cardiomyocyte reference genes.

SDS-PAGE of Myosin Heavy Chain Isoforms. A 6.25% acrylamide/bis-acrylamide(99:1) resolving gel is prepared by combining 7.5 mL of 25% Acrylamide/bis-acrylamide(99:1), 5.65 mL of 2M Tris pH 8.8, 16.55 mL of ddH20, 300 μl of 10% SDS (w/v), 312 μl 10% ammonium persulfate, and 12.5 μl of TEMED. The resolving gel is allowed to polymerize for 1 hour at room temperature. A 5% acrylamide/bis-acrylamide (99:1) stacking gel is prepared by combining 2 mL of 25% Acrylamide/bis-acrylamide(99:1), 2.5 mL of 0.5M Tris pH 6.8, 5.325 mL of ddH20, 100 μl of 10% SDS (w/v), 90 μl 10% ammonium persulfate, and 6 μl of TEMED. The stacking gel is allowed to polymerize for 8 hours. Lysates of approximately day 20 iPSC-CMs are prepared and protein concentrations are quantified with the Quick-Start Bradford Protein Assay (Bio-Rad). approximately 7 μg of protein is mixed 1:1 with 2× Laemmli Sample Buffer containing β-mercaptoethanol. Samples are loaded into the SDS-polyacrylamide gel described above and separated at 13 mA for 20 min, and 15 mA for 21 hours. After electrophoresis, gels are fixed with a 7% acetic acid/50% methanol solution for 1 hour at room temperature. Protein is visualized with the Sypro Ruby Protein Gel Stain (Thermo Fisher) following manufacturer's instructions. Quantification of band intensities is done using Fiji³⁵.

### Engineered Heart Tissue Generation and Measurement of Contractile Properties.

Engineered heart tissues (EHTs) are generated according to previously published methods³⁶. iPSC-CMs are differentiated as previously described and when beating cells are present (approximately day 10), cells are washed with PBS and digested with TrypLE (Thermo). One million cells per EHT are centrifuged at 500 g for 5 min and resuspended in 65 μl of EHT media (CDM3³², containing 10% of heat-inactivated FBS, 2 μM thiazovivin, 33 μg/mL aprotinin, and 5 U/mL penicillin/streptomycin), 25 μl of 25 mg/mL fibrinogen and 10 μl of Matrigel (Corning). 100 μl of this EHT mix is added to 3 μl of 100 U/mL thrombin and mixed. The whole mixture is pipetted between PDMS posts (EHT Technologies) in an EHT mold created from 2% agarose and a Teflon spacer in a 24-well Nunc plate (Thermo Fisher). Fibrin gel is allowed to polymerize for 2 hours and then 200 μl of CDM3 is added to the EHT to help detach it from the mold. After 30 min, the PDMS posts are lifted from the mold and the EHT is placed into a new 24 well plate containing 1.6 mL of RPMI containing B27 supplement (Thermo Fisher) and 33 μg/mL aprotinin. Media is changed every other day until further processing. After 20 days of culture, videos of EHT contraction are taken on a KEYENCE BZ-X microscope at 50 fps with 4×4 pixel binning. Videos are imported into Fiji and analyzed with MUSCLEMOTION macro with default settings³⁷. The contraction parameters for each contraction are averaged to give an EHT level measurement.

Flow Cytometry Analysis of IPSC-CM Purity. At approximately day 20 of differentiation, iPSC-CMs are collected using TrypLE (Thermo Fisher). Cells are resuspended in 1 mL of PBS and added to 1 mL of 8% PFA in PBS for fixing. Cells are fixed at 37° C. for 10 min with shaking. Cells are collected by centrifugation at 600 g for 5 min and resuspended in 100 μl ice-cold 90% methanol in PBS per 500,000 starting cells. Cells are stored at −20° C. until further processing. On the day of flow, approximately 1 million cells are aliquoted into two tubes containing 2 mL of 0.5 mg/mL BSA in PBS and pelleted. One tube is resuspended in 100 μl of PBS containing 1:200 dilution of TNNT2-Alexa Fluor 694 (BD Pharmingen #565744) and 1:200 MYBPC3-Alexa Fluor 488 (Santa Cruz Biotechnology #sc-137180 AF488) and the other tube is suspended in PBS alone. Cells are stained for 1 hour at room temperature. Four mL of 0.5 mg/mL BSA in PBS is added to each tube and cells are pelleted. Cells are resuspended in 100 μl in PBS and analyzed on a flow cytometer. The percentage of TNNT2-positive cells is determined by using PBS only as a negative control.

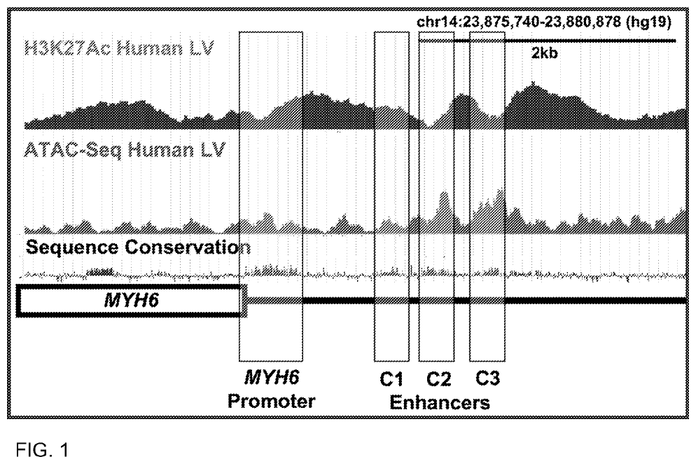

Find Regulatory Variants Computational Pipeline. FIG. 1 shows a schematic of the Find Regulatory Variants computational pipeline. The pipeline relies on the bedtools tool to sequentially filter the starting variant list for variants that overlap regions with epigenetic evidence of enhancer modifying potential³⁰. The epigenetic datasets were all derived from human left ventricle, mouse left ventricle, HL-1 cardiomyocytes, and IPSC-CMs.

In datasets where multiple replicates were available, a superset representing all peaks found was created. The pipeline finds variants that are predicted to disrupt or create transcription factor binding sites. In order to find new transcription factor binding sites created by variants, the GATK FastaAlternateReferenceMaker will be used to insert SNP variants into the reference genome³⁸. Homer's scanMotifGenomeWide.pl will then be used to search for GATA4 and TBX5 sites in the alternative reference and kept only sites that were new³⁹. In the case of multi-allelic variants, one alternative allele are chosen at random. These additional sites are used in the pipeline alongside sites present in the unchanged reference. This pipeline was executed on variants that passed all quality filters from the gnomAD v.2.1 release.

Association of Enhancer Variant with Phenotypic Data. Phenotypic measurements of heart function and whole genome sequencing data are accessed as in²¹. Individual measures are obtained for left ventricular internal diameter-diastole (LVIDd) and left ventricular posterior wall thickness during diastole (LVPWd) from echocardiogram reports and spanned as much as 14 years of echocardiogram data. The diagnosis of heart failure is determined by ICD9 diagnosis codes 425 and all sub-codes, and ICD10 diagnostic codes I42 and all sub-codes. Trajectory analysis of echo measurements is conducted as in²¹. Briefly, PROC TRAJ in SAS 9.4 is used,⁴⁰ which uses a likelihood function to assign a each individual a phenotypic cluster and probability of belonging to that cluster.

### Example 2—Integrated Epigenetic Analysis Identifies Candidate Enhancer Regions for MYH6

To find putative modifying regulatory variants associated with heart failure, the regulatory landscape of MHY6 was characterized. To identify enhancer regions active in the human left ventricle, multiple datasets including human left ventricle-derived H3K27Ac ChIP-seq and ATAC-seq, as well as ChIP-seq data of genome-wide binding of the cardiogenic transcription factors GATA4, TBX3/5, and NKX2.5 were overlaid from multiple cell/tissue sources. Promoter-capture Hi-C data from iPSC-CMs was used to identify genomic regions predicted to interact with promoters¹⁰. Intersection of these datasets identified three enhancer clusters for MYH6 (FIG. 1).

### Example 3—Candidate MYH6 Enhancers and Regulatory Activity in Cardiomyocytes

Next, using a luciferase reporter assay, the regulatory potential of the candidate enhancer regions identified in the MYH6 is determined experimentally. Promoter-capture Hi-C data is used to define the boundaries of individual enhancers within clusters. Three MYH6 enhancer regions are tested to determine activity in iPSC-CMs compared to a negative control genomic desert region.

### Example 4—Deletion of MYH6 Enhancer Regions and Effect on MYH7 to MYH6 Protein Levels

To test if candidate enhancers are required for target gene expression, regions of interest in iPSCs are deleted using gene editing. A dual cutting CRISPR-Cas9 strategy is employed to remove the candidate enhancer regions (i.e., MYH6-C1, MYH6-C2, and MYH6-C3). PCR genotyping will be used to confirm the expected heterozygous and homozygous deletion in independent lines.

Enhancer-deleted iPSCs will be differentiated into cardiomyocytes and MYH7 and MYH6 mRNA expression will be measured using qPCR.

### Example 5—Active Cardiac Enhancers Harbor Genetic Variants in Transcription Factor Binding Sites

Next, the MYH6 enhancers will be characterized for naturally occurring sequence variants using the gnomAD database and those that overlap cardiac transcription factor binding motifs, and/or were correlated with MYH6 expression in the GTEx eQTL dataset^{15,16} will be selected.

### Example 6—CRISPR Activation is Sufficient to Drive MYH6 Expression in a Non-Cardiomyocyte Environment

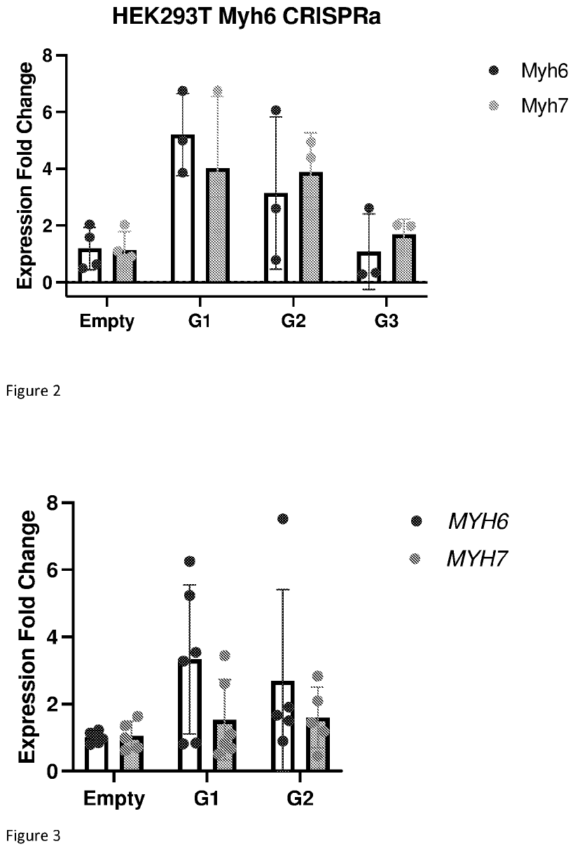

Three single guide RNAs (sgRNAs) were designed within the region labeled as candidate enhancer region labelled C2 in FIG. 2A in Gacita et al., 2021 PMCID: PMC8009836. The sgRNAs were ligated into a plasmid compatible with both adeno-associated virus packaging or transient transfection, pAAV6.CMV.dSaCas9-VP64.U6.[sgRNA/empty], which is publicly available from Addgene. Guide sequences are listed as follows: G1: GCCCCACCCTCTGTCTAAATT (SEQ ID NO: 1, Distance from TSS: −35; Specificity score: 76); G2: TCATCAGCCCTTTGAAGGGG (SEQ ID NO: 2, distance from TSS: −232; Specificity score: 72); G3: TCCTGTCACCTCCAGAGCCA (SEQ ID NO: 3; Distance from TSS: −119; Specificity score: 57).

HEK293T cells were seeded at a density of 500,000 cells per well into two 6-well plates and allowed to attach for 24 hours in Dulbecco's Modified Eagle Medium/Nutrient Mixture F-12+10% qualified fetal bovine serum. Using Lipofectamine 3000 (ThermoFisher), multiple wells of cells were then transfected with one each of the 3 sgRNAs, and other wells with empty plasmid (negative control), and a single well with plasmid pGFPmax (positive transfection control). Cells were exposed to transfection reagent and plasmid for six hours at which time, the cells were then cultured in standard media. Twenty four hours after transfection, cells were harvested, and RNA was isolated using TRIzol. cDNA was generated using qScript cDNA SuperMix (Quantabio). Quantitative polymerase chain reaction with SYBR Green (ThermoFisher) was used to assay MYH6 and MYH7 expression using the following primers: MYH6 Forward: AAGTCCTCCCTCAAGCTCATGGC (SEQ ID NO: 4); MYH6 Reverse: ATTTTCCCGGTGGAGAGC (SEQ ID NO: 5); MYH7 Forward: GCAGCTAAAGGTCAAGGCC (SEQ ID NO: 6); MYH7 Reverse: AGCTACTCCTCATTCAAGCC (SEQ ID NO: 7).

Results: Derivations of CRISPR machinery can be used to drive gene expression by fusing a catalytically dead Cas9 with a transactivator like VP64. Introduction of the dCas9-VP64 fusion protein alongside a sgRNA to engage specific sequences can drive gene expression, provided the sequences are regulatory regions. Candidate regulatory regions for MYH6 were identified and sgRNAs were designed to be homologous to regulatory regions with the C2 domain as defined in Gacita et al. Plasmids expressing specific sgRNAs along with dCas9-VP64 were introduced into HEK293T cells to target the MYH6 promoter and drive MYH6 gene expression. HEK293T cells transfected with guide 1 showed the largest increase in MYH6 expression, while cells transfected with guide 2 and guide 3 showed modest increases in MYH6 expression when compared to control plasmid containing an empty sgRNA sequence as indicated by RT-qPCR. See FIG. 2. MYH6 and MYH7 are known to share regulatory elements, so some MYH7 upregulation was also observed in this non-cardiomyocyte cell line.

### Example 7—CRISPR Activation Upregulated MYH6 in hiPSC-CMs, a Human Cardiomyocyte Environment

hiPSCs (Coriell Institute, GM03348, healthy male control) were plated onto 6 well plates coated in Matrigel (Corning) and differentiated into hiPSC-CMs as described the differentiation protocol using CHIR99021 and WNT-C59. On day 10 of differentiation, hiPSC-CMs were enriched using non-CM depletion via the Miltenyi Biotec PSC-Derived Cardiomyocyte Isolation Kit MACS system and replated at a density of 700,000 hiPSC-CMs per well in 9 wells of a Matrigel-coated 12 well plate. Three wells each per guide 1, guide 2, and empty plasmid negative control were transfected on day 8 following replating using Lipofectamine 3000 per manufacturer's instructions. Transfection reagent was removed 24 hours later, and cells were harvested for RNA isolation. RNA isolation and quantitative polymerase chain reaction was performed as described above in Example 6.

Results: To assess whether these sgRNAs could selectively increase MYH6 expression a human cardiomyocyte cell, the same assay was performed in hiPSC-CMs using the two sgRNAs with the largest effect (guide 1 and guide 2). hiPSC-CMs transfected with guide 1 showed a significant increase in MYH6 expression and hiPSC-CMs transfected with guide 2 showed a modest increase in MYH6 expression. MYH7 gene expression was not increased (as assessed by RT-qPCR), indicating specificity of this gene expression for MYH6 in the human cardiomyocyte context. See FIG. 3.

### REFERENCES

1. McNally, et al., J Clin Invest 123, 19-26 (2013).
2. Konno, T., et al., Curr Opin Cardiol 25, 205-9 (2010).
3. Maron, et al., JACC Heart Fail 6, 376-378 (2018).
4. McNally, et al., Circ Res 121, 731-748 (2017).
5. Marian, A. J. Curr Opin Cardiol 17, 242-52 (2002).
6. Barp, A. et al. PLoS One 10, e0141240 (2015).
7. Verdonschot, J. A. J. et al. Mol Genet Genomic Med 8, e1049 (2020).
8. Andersson, et al., Nat Rev Genet 21, 71-87 (2020).
9. Tanjore, R. et al., Indian J Hum Genet 16, 67-71 (2010).
10. Montefiori, L. E. et al., Elife 7(2018).
11. Morkin, E., Circulation 87, 1451-60 (1993).
12. Leung, D. et al., Nature 518, 350-354 (2015).
13. Visel, et al., Nucleic Acids Res 35, D88-92 (2007).
14. VanBuren, et al., Circ Res 77, 439-44 (1995).
15. Karczewski, et al., bioRxiv, 531210 (2020).
16. Consortium, G. T., Nat Genet 45, 580-5 (2013).
17. Lundquist, M. R. et al., Cell 156, 563-76 (2014).
18. Houweling, et al., Cardiovasc Res 67, 583-93 (2005).
19. Hershberger, R. E. et al., Circ Cardiovasc Genet 2, 306-13 (2009).
20. Yilbas, A. E. et al., Front Chem 2, 12 (2014).
21. Pottinger, T. D. et al., bioRxiv, 2020.05.10.087130 (2020).
22. Pott, S. et al., Nat Genet 47, 8-12 (2015).
23. Hnisz, D. et al., Cell 155, 934-47 (2013).
24. Man, J. C. K. et al., Nat Commun 10, 4943 (2019).
25. Nakao, et al., J Clin Invest 100, 2362-70 (1997).
26. Abraham, W. T. et al., Mol Med 8, 750-60 (2002).
27. Tripathi, S. et al., Basic Res Cardiol 106, 1041-55 (2011).
28. Jiang, J., et al., Science 342, 111-4 (2013).
29. Dickel, D. E. et al., Nat Commun 7, 12923 (2016).
30. Quinlan, et al., Bioinformatics 26, 841-2 (2010).
31. Claycomb, et al., Proc Natl Acad Sci USA 95, 2979-84 (1998).
32. Burridge, et al., Nat Methods 11, 855-60 (2014).
33. Kim, E. Y. et al., JCI Insight 4(2019).
34. Haeussler, et al., Genome Biol 17, 148 (2016).
35. Schindelin, J. et al., Nat Methods 9, 676-82 (2012).
36. Schaaf, S. et al., Methods Mol Biol 1181, 121-9 (2014).
37. Sala, L. et al., Circ Res 122, e5-e16 (2018).
38. McKenna, A. et al., Genome Res 20, 1297-303 (2010).
39. Heinz, S. et al., Mol Cell 38, 576-89 (2010).
40. Jones, et al., Sociological Methods & Research 35, 542-571 (2007).
41. Roadmap Epigenomics, C. et al., Nature 518, 317-30 (2015).
42. Davis, C. A. et al., Nucleic Acids Res 46, D794-D801 (2018).
43. Liu, Q. et al., Circ Res 121, 376-391 (2017).
44. May, D. et al., Nat Genet 44, 89-93 (2011).
45. Ang, Y. S. et al., Cell 167, 1734-1749 e22 (2016).
46. van den Boogaard, M. et al., J Clin Invest 122, 2519-30 (2012).
47. He, et al., Proc Natl Acad Sci USA 108, 5632-7 (2011).
48. Gacita, et al., bioRxiv, 2020.03.17.988790 (2020).

What is claimed is:

1. A method for increasing expression of the myosin heavy chain 6 (MYH6) gene in a cell comprising introducing into the cell one or more DNA endonucleases and one or more guide RNAs that target a region within chr14:23877406-23879693 as designated in the human genome browser, build 37 (hg37), of the MYH6 gene that results in increased expression of the MYH6 gene.

2. The method of claim 1, that results in activation of an enhancer region of the MYH6 gene in the cell, relative to a cell into which the DNA endonuclease was not introduced.

3. The method of claim 2, wherein the enhancer region is upstream of the MYH6 gene.

4. The method of claim 2 or claim 3, wherein the enhancer region is MYH6-C1, MYH6-C2, or MYH6-C3.

5. The method of any one of claims 1-4, that results in decreased myosin heavy chain 7 (MYH7) expression and increased MYH6 expression in the cell, relative to a cell into which the DNA endonuclease was not introduced.

6. The method of any one of claims 1-5, that results in an increased speed of contraction in the cell, relative to a cell into which the DNA endonuclease was not introduced.

7. The method of any one of claims 1-6, wherein the DNA endonuclease is Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 or a homolog thereof.

8. The method of any one of claims 1-6, wherein the DNA endonuclease is a nuclease defective DNA endonuclease.

9. The method of claim 8, wherein the DNA endonuclease is dCas9.

10. The method of claim 9, wherein the dCas9 is fused to a transcriptional modulator.

11. The method of claim 10, wherein the transcriptional modulator is a transcriptional activator.

13. The method of claim 11, wherein the transcriptional activator is tetracycline transactivator, VP16, VP64, synergistic activation mediator, SunTag.

14. The method of claim 10, wherein the transcriptional modulator is a transcriptional suppressor.

15. The method of any one of claims 1-14, wherein the method comprises introducing into the cell one or more polynucleotides encoding the one or more DNA endonucleases.

16. The method of any one of claims 1-13, wherein the method comprises introducing into the cell one or more ribonucleic acids (RNAs) encoding the one or more DNA endonucleases.

17. The method of any one of claims 1-13, wherein the one or more gRNAs comprises a nucleotide sequence set forth in SEQ ID NO: 1, SEQ ID NO: 2 or SEQ ID NO: 3 or combinations thereof.

18. The method of any one of claims 1-17, wherein the one or more DNA endonucleases is pre-complexed with one or more gRNAs.

19. The method of any one of 1-18, wherein the DNA endonuclease and one or more guide RNAs are delivered by a viral vector.

20. The method of claim 19, wherein the viral vector is a herpes virus vector, an adeno-associated virus (AAV) vector, an adeno virus vector, or a lentiviral vector.

21. The method of claim 19, wherein the viral vector is an adeno-associated virus (AAV) vector.

22. The method of claim 21, wherein the AAV vector is recombinant AAV5, AAV6, AAV8, AAV9, or AAV7.

23. The method of any one of claims 19-22, wherein the vector further comprises a cardiac tissue specific promoter.

24. The method of claim 23, wherein the cardiac the tissue-specific promoter is TNNT2, MLC2v, creatine kinase (CK), and derivatives thereof.

25. The method of any one of claims 1-24, wherein the DNA endonuclease and/or gRNAs are delivered by a lipid nanoparticle (LNP).

26. A method of improving cardiac contractility in a subject in need thereof comprising administering to the subject an agent that increases myosin heavy chain 6 (MYH6) gene expression in a cardiac cell from the subject.

27. The method of claim 26, wherein the agent also decreases myosin heavy chain 7 (MYH7) gene expression in a cardiac cell of the subject.

28. The method of claim 26 or claim 27, wherein the subject is suffering from heart failure.

29. The method of any one of claims 26-28, wherein the agent is one or more DNA endonucleases and one or more guide RNAs that target a region within chr14:23877406-23879693 as designated in the human genome browser, build 37 (hg37) of the MYH6 gene that results in activation of an enhancer region of the MYH6 gene.

30. The method of claim 29, wherein the DNA endonuclease is Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 or a homolog thereof.

31. The method of claim 29 or claim 30, wherein the DNA endonuclease is a nuclease defective DNA endonuclease.

32. The method of claim 31, wherein the nuclease defective DNA endonuclease is dCas9.

33. The method of claim 32, wherein the dCas9 is fused to a transcriptional modulator.

34. The method of claim 33, wherein the transcriptional modulator is a transcriptional activator.

35. The method of claim 34, wherein the transcriptional activator is tetracycline transactivator, VP16, VP64, synergistic activation mediator, SunTag.

36. The method of claim 33, wherein the transcriptional modulator is a transcriptional suppressor.

37. The method of claim 26, wherein the enhancer region is upstream of the MYH6 gene.

38. The method of claim 37, wherein the enhancer region is MYH6-C1, MYH6-C2, or MYH6-C3.

39. The method of any one of claims 26-38 that results in decreased myosin heavy chain 7 (MYH7) expression and increased MYH6 expression in the cell, relative to a cell into which the Cas9 was not introduced.

40. The method of any one of claims 29-39, wherein the one or more gRNA comprises a nucleotide sequence set forth in SEQ ID NO: 1, SEQ ID NO: 2 or SEQ ID NO: 3, or combinations thereof.

41. The method of any one of claim 29-40, wherein the DNA endonuclease and one or more guide RNAs are delivered by a viral vector.

42. The method of claim 41, wherein the viral vector is a herpes virus vector, an adeno-associated virus (AAV) vector, an adeno virus vector, or a lentiviral vector.

43. The method of claim 42, wherein the viral vector is an adeno-associated virus (AAV) vector.

44. The method of claim 43, wherein the AAV vector is recombinant AAV5, AAV6, AAV8, AAV9, or AAV7.

45. The method of any one of claims 42-44, wherein the vector further comprises a cardiac tissue specific promoter.

46. The method of claim 45, wherein the cardiac tissue-specific promoter is TNNT2, MLC2v, creatine kinase (CK), and derivatives thereof.

47. The method of any one of 29-46, wherein the DNA endonuclease and one or more guide RNAs are delivered by lipid nanoparticles (LNPs).


SEQUENCE LISTING (7 sequences)

SEQ ID NO: 1 - DNA, 21 bp, synthetic construct
  source 1..21: mol_type = other DNA
gccccaccctctgtctaaatt

SEQ ID NO: 2 - DNA, 20 bp, synthetic construct
  source 1..20: mol_type = other DNA
tcatcagccctttgaagggg

SEQ ID NO: 3 - DNA, 20 bp, synthetic construct
  source 1..20: mol_type = other DNA
tcctgtcacctccagagcca

SEQ ID NO: 4 - DNA, 23 bp, synthetic construct
  source 1..23: mol_type = other DNA
aagtcctccctcaagctcatggc

SEQ ID NO: 5 - DNA, 18 bp, synthetic construct
  source 1..18: mol_type = other DNA
attttcccggtggagagc

SEQ ID NO: 6 - DNA, 19 bp, synthetic construct
  source 1..19: mol_type = other DNA
gcagctaaaggtcaaggcc

SEQ ID NO: 7 - DNA, 20 bp, synthetic construct
  source 1..20: mol_type = other DNA
agctactcctcattcaagcc